(19) United States
(12) Patent Application Publication
(10) Pub. No.: US 2001/0031463 A1
(43) Pub. Date: Oct. 18, 2001

(54) Method for purifying and concentrating AAV-2 and antigen portions thereof

(76) Inventors: Jurgen Kleinschmidt, Bammental (DE); Andrea Kern, Ittlingen (DE)

Correspondence Address: HOWREY SIMON ARNOLD & WHITE, LLP, BOX 34, 301 RAVENSWOOD AVE., MENLO PARK, CA, 94025, US

(21) Appl. No.: 09/886,144

(22) Filed: Jun. 20, 2001

Related U.S. Application Data
(63) Continuation of application No. 09/508,037, filed on Jun. 23, 2000, now abandoned.
Application No. 09/508,037 is a 371 of international application No. PCT/DE98/02569, filed on Sep. 1, 1998.

(30) Foreign Application Priority Data
Sep. 2, 1997 (DE) 197 38 292.4

(51) Int. Cl. C12Q 1/70; G01N 33/53; C12P 21/08; C07K 1/00; C07K 16/00

(52) U.S. Cl. 435/5; 436/515; 436/516; 435/7.1; 530/388.3; 530/826

(57) ABSTRACT

The invention relates to a method for purifying and concentrating AAV-2 and antigen portions thereof. AAV-2 or antigen portions thereof are bonded to an activated chromatographic material on which antibodies are linked, said antibodies being directed against AAV-2. Afterwards, elution using a solution containing 0.5 to 4.5 M MgCl₂ is carried out. The invention also relates to a kit for implementing said method.

The present invention relates to a method for purifying AAV-2 and antigen portions thereof.

For therapeutic approaches viruses, particularly recombinant viruses which are used as vectors, must be purified and concentrated. The methods used for this purpose so far, such as multiple density gradient centrifugation or chromatographic methods, e.g. with a sulfonated cellulose column, are often complicated, time-consuming and expensive and must be repeated several times to remove as many contaminations as possible. Nevertheless, only partial purification is often possible by this.

Therefore, it is the object of the present invention to provide a method by which adeno-associated type 2 viruses (AAV-2) can be purified and concentrated simply and efficiently from cell culture supernatants and cell extracts.

This object is achieved by the subject matters defined in the claims.

In particular, this object is achieved by a method for purifying and concentrating AAV-2 and antigen portions thereof, which comprises the steps of:

linking an antibody directed against AAV-2 to an activated chromatographic material,

applying a sample containing wt-AAV-2 and rAAV-2, respectively,

eluting wt-AAV-2 and rAAV-2, respectively, with a solution, preferably a buffer containing 0.5 to 4.5 M MgCl₂.

The method according to the invention is based on the principle of affinity chromatography. In a preferred embodiment, an antibody directed against AAV-2 is bonded to chromatographic material. The antibody is bonded to the chromatographic material according to known methods, e.g. as described in “Harlow and Lane, Antibodies (A Laboratory Manual), Cold Spring Harbor, 1988”. The chromatographic materials known to a person skilled in the art, particularly the hydrophilic materials which are known in connection with gel filtration, are suitable as chromatographic material. These are e.g. agarose gels (e.g. sepharose^{R}), dextran gels (e.g. sephadex^{R}), cellulose gel matrices and acrylamide gel matrices. For the purpose according to the invention the chromatographic material is advantageously activated, so as to enable the linkage and immobilization of proteins. This activation can take place by cyanogen bromide (CNBr) activation or NHS activation.

The antibodies usable for the method according to the invention recognize AAV-2 or antigen portions thereof. Such an antibody can be a polyclonal or monoclonal antibody. The antibody can be obtained from any animal or a human being, rabbits being preferred for a polyclonal antibody and mice being preferred for a monoclonal antibody. The antibody may also be synthetic, portions which are not necessary for the above recognition optionally lacking fully or partially therefrom and these portions being replaced by others which provide the antibody with further favorable properties, respectively. Portions can also be modified, eliminated or replaced outside the binding regions of the antibodies. The person skilled in the art knows that for the above measures the DNA recombination technique is particularly suitable. He is perfectly familiar therewith. For the method according to the invention particularly the previously known antibody is suitable which is described in WO 95/11997 and EP-A-0 725 837, respectively: A20; deposited with the DSMZ (Deutsche Sammlung für Mikroorganismen und Zellkulturen) [German-type collection of microorganisms and cell cultures], under DSM ACC2194 on Oct. 13, 1994.

The expression “AAV-2 or antigen portions thereof” relates to AAV-2 capsid proteins, particularly VP1, VP2 and/or VP3 as well as the fragments thereof which comply with an antigen function. The expression “AAV-2” comprises both wild-type and recombinantly produced AAV-2. Detailed information as regards methodic aspects of AAV-2, such as cell culture, viral growth, preliminary viral purification, isolation of the proteins can be found in the relevant literature, e.g. in Handbook of Parvoviruses, Vols. I and II, CRC Press, Boca Raton, Fla., ed. P. Tjissen; Ruffing, M. et al. (1992), J. Virol. 66, pp. 6922-6930.

If one places on a chromatographic column to which an antibody directed against AAV-2 is linked, a solution containing AAV-2, e.g. in the form of cell culture supernatants or cell extracts, bonding to the immobilized antibody will take place and AAV-2 can be separated therefrom again, so that purification and concentration is effected. In this connection, the elution conditions are within the almost neutral range, about pH 6 to 8. The elution solution contains 0.5 to 4.5 M MgCl₂, preferably 2 to 3 M MgCl₂. These conditions have the advantage that the immobilized antibody is not denatured and not separated from the column during the elution of AAV-2 but continues to be bonded to the column, so that the column can be used several times after its regeneration. Furthermore, the purified viruses remain stable and infectious, respectively, by the method according to the invention.

In a preferred embodiment, the activated chromatographic material can also contain spacers bound thereto. These spacers are preferably aliphatic or cyclic hydrocarbon groups. The aliphatic hydrocarbon groups can be straight-chain or branched ethyl, propyl, butyl, pentyl, hexyl, heptyl, octyl, nonyl, or decyl groups. The cyclic hydrocarbon groups to be mentioned are phenyl, benzyl, cyclohexyl groups.

For carrying out the method according to the invention a kit is also provided which also represents a subject matter of the present invention. Such a kit comprises the following:

an antibody directed against AAV-2, and

conventional auxiliary agents, such as buffers,

chromatographic matrices and controls.

By means of the method according to the invention great purification is obtained by a single purification step. In this connection, complete removal of helper viruses (adenoviruses) is possible. The concentration from large volumes is also possible, a yield of almost 100 % being achievable. For great outputs, scale-up of the method according to the invention is possible in a simple manner.

The invention is further described by means of the figures, which show:

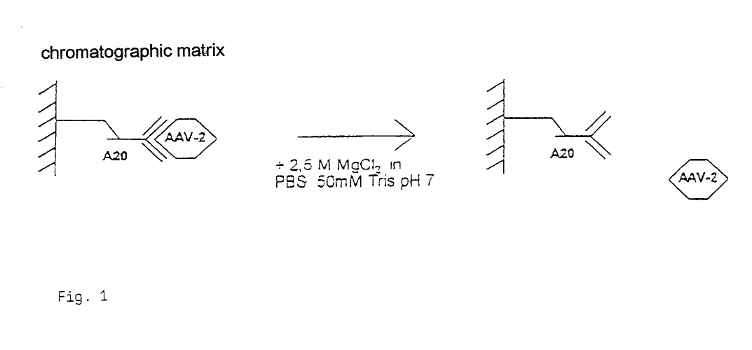

FIG. 1: principle of the method according to the invention

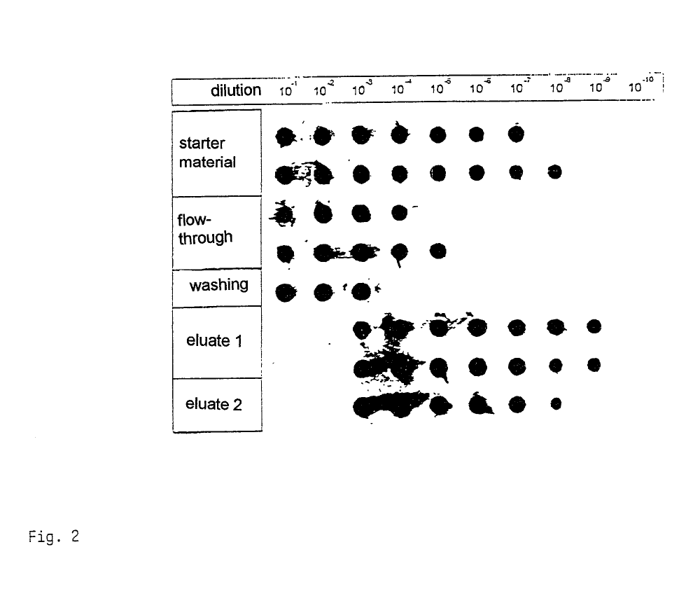

FIG. 2: dot blot

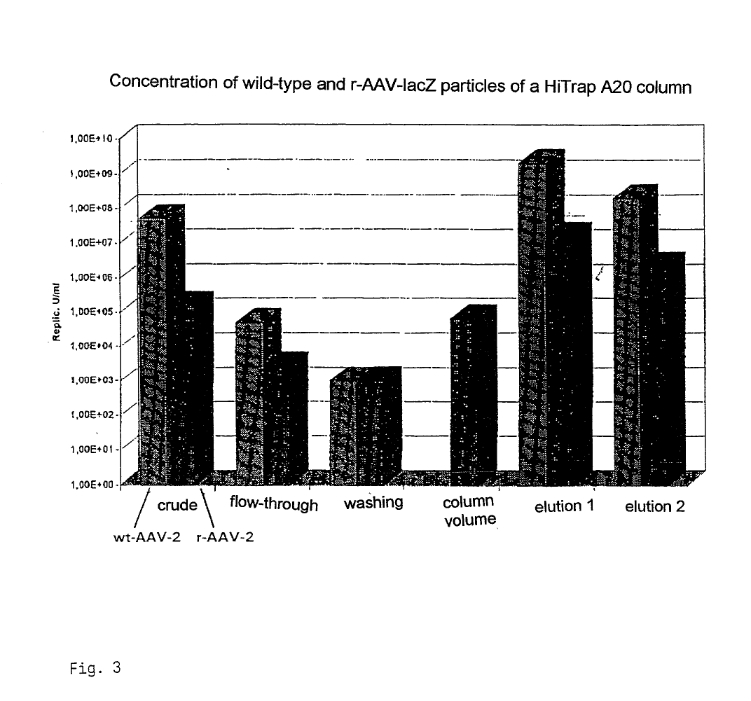

FIG. 3: diagram of the elution (light gray columns: wild-type AAV-2 ; dark gray columns: rAAV-2).

The invention is further described by means of the following examples:

### EXAMPLE 1

The method is shown in principle in FIG. 1.

About 8 mg A20 antibodies were affinity-purified from 500 ml A20 hybridoma supernatant through an anti-mouse IgG column (a “Quick Antibody Matrix” from Dianova company can also be used as an alternative). 8 mg A20 antibodies were linked to HiTrap NSH-activated sepharose (Pharmacia company) in accordance with the manufacturer's instructions. A clarified freeze-thaw lysate of cells in which wild-type AAV-2 or rAAV-2 was produced, was slowly pumped through the column (4° C. overnight), washed with 10 column volumes of PBS and eluted in 2 to 4 ml PBS using 2.5 M MgCl₂. MgCl₂ can be removed by dialysis or pelleting of the viral particles and resuspension in a desired buffer (e.g. Tris or 10/1 TE).

### EXAMPLE 2

The A20-monoclonal antibody was purified and concentrated from the hybridoma supernatant using the “Quick Antibody Purification Column” from Dianova company, Hamburg. The purified antibody was linked to HiTrap-NHS-activated sepharose according to the instructions from the manufacturer (Pharmacia company). An unpurified AAV-2 viral preparation was diluted at a ratio of 1:10 with sterile PBS and slowly applied onto the A20-HiTrap column. Following the collection of the flow-through, the column was washed with PBS and the AAV-2 capsids were eluted with 2.5 M MgCl₂ in PBS, completed with 50 mM Tris, pH 7. The A20-HiTrap column was regenerated using PBS and stored under PBS/0.01% sodium azide.

The AAV-2 viruses were titrated by dot blot hybridization and immunofluorescence. For this purpose, HeLa cells were filled into 96-well microtiter plates in a concentration of about 1×10⁴ cells/well, infected with AAV-2 from various fractions of the above affinity experiment (material applied, flow-through, wash solution, elution fraction) in serial dilutions and overinfected with type 5 adenovirus (MOI=5). After three days, the cells were fixed for the immunofluorescence or frozen/thawed for the dot blot hybridization (3 times). The infection with AAV-2 was observed by immunofluorescence with the monoclonal antibody 76/3 (Progen company, Heidelberg) for the detection of the AAV-2 rep gene expression. Replicating AAV-2 were detected with a labeled DNA fragment of the AAV-2 Rep gene (dots in FIG. 2; light gray columns in FIG. 3).

Recombinant AAV-2 was also purified according to the above method for wild-type AAV-2. For the titration of rAAV-2, the expression of the LacZ gene was measured by in situ X-Gal staining of the infected HeLa cells according to serial dilutions (dark gray columns in FIG. 3).

1. A method for purifying and concentrating AAV-2 and antigen portions thereof, characterized in that AAV-2 or antigen portions thereof are bonded to an activated chromatographic material which comprises antibodies linked thereto and directed against AAV-2, and then elution is carried out using a solution containing 0.5 to 4.5 M MgCl₂.

2. The method according to claim 1, wherein AAV-2 is either wild-type AAV-2 or recombinantly prepared AAV-2.

3. The method according to claim 1 or 2, wherein the chromatographic material is selected from the group consisting of agarose gels, dextran gels, cellulose gel matrices and acrylamide gel matrices.

4. The method according to any one of claims 1 to 3, wherein the chromatographic material carries a ligand suitable for bonding proteins, particularly antibodies.

5. The method according to any one of claims 1 to 4, wherein the chromatographic material is CNBr-activated sepharose^{R} or NHS-activated sepharose^{R}.

6. The method according to any one of claims 1 to 5, wherein the elution solution contains 2 to 3 M MgCl₂.

7. The method according to any one of claims 1 to 6, wherein the sample containing the AAV-2 and rAAV-2, respectively, is a cell culture supernatant or cell extracts.

8. The method according to any one of claims 1 to 7, wherein the antibody directed against AAV-2 is A20 (DSM ACC2194).

9. A kit for carrying out the method according to any one of claims 1 to 8 comprising
an antibody directed against AAV-2, and
conventional auxiliary agents, such as buffers, chromatographic material and controls.
